(19) United States
(12) Patent Application Publication
(10) Pub. No.: US 2003/0068621 A1
(43) Pub. Date: Apr. 10, 2003

(54) Method and device for producing oligonucleotide arrays

(76) Inventor: Jonathan Briggs, Los Altos Hills, CA (US)

Correspondence Address: PILLSBURY WINTHROP, LLP, P.O. BOX 10500, MCLEAN, VA, 22102, US

(21) Appl. No.: 09/969,887

(22) Filed: Oct. 4, 2001

(51) Int. Cl. C12Q 1/68; C12M 1/34; B05D 3/00

(52) U.S. Cl. 435/6; 435/287.2; 427/2.11

(57) ABSTRACT

The present invention relates to a method for producing a nucleic acid, polynucleotide, oligonucleotide, or polymer array and device therefore, wherein the method includes synthesizing a plurality of positionally addressable nucleic acids, polynucleotides, oligonucleotides, or polymers in a reaction array, and transferring these positionally addressable substances from the reaction array directly to a substrate to form the desired product array.

### FIELD OF THE INVENTION

This invention relates to the manufacture and use of nucleic acids, nucleic acid arrays, polynucleotides, polynucleotide arrays, oligonucleotides, oligonucleotide arrays, polymers, and polymer arrays. More particularly, this invention relates to an improved process and device for producing oligonucleotide arrays.

### BACKGROUND OF THE INVENTION

At present, there are at least two common techniques for manufacturing oligonucleotide product arrays: (1) spotting pre-synthesized polynucleotides (mainly cDNA) onto the desired substrate; and (2) synthesizing polynucleotides in situ on an array substrate.

Spotted arrays are generally assembled by placing a pre-synthesized oligonucleotide onto a substrate. This approach can require a library of pre-synthesized oligonucleotides, however, and can suffer from low spot-to-spot uniformity due to the relatively uncontrolled process by which the DNA is deposited onto the substrate. In addition, the positional complexity required to organize, store, and track thousands of pre-synthesized oligonucleotides during the manufacturing of the array results in a multitude of manufacturing and quality control issues.

In contrast to the spotted technique described above, the in situ techniques build the desired oligonucleotide directly on the array substrate thereby avoiding many of the organization, tracking, and storing complexities associated with the spotting techniques. Such in situ methods are exemplified in the photolithographic techniques developed by Affymetrix, Inc. (Santa Clara, Calif.) wherein selective irradiation of the chip surface directs the stepwise attachment of each base of the desired oligonucleotide.

Other attempts at in situ synthesis have centered on directly delivering one of the four phosphoramidite reagents to designated sites on the array via a printing process. There is always a need for ways to streamline the production of oligonucleotide arrays to facilitate high volume chip production for diagnostic applications, while also providing flexibility to tailor chip production to lower volume needs, i.e., as in discovery applications.

Accordingly, one advantage of the present invention provides an improved method and device by which arrays of nucleic acids, polynucleotides, oligonucleotides, and polymers may be efficiently assembled. In particular, the invention relates to nucleic acid, polymer, oligomer and oligonucleotide arrays. The present invention provides these improvements by offering the industry a simplified synthesis and tracking mechanism which provides for the ability to produce custom synthesized polynucleotides and polymers (for flexibility of design), which may be covalently linked to the reaction sites of the array (for good bio-accessibility during hybridization).

### SUMMARY OF THE INVENTION

The present invention is a method for producing nucleic acid, polymer, oligomer or oligonucleotide array comprising: synthesizing a set of positionally addressable nucleic acids, polymers, oligomers or oligonucleotides in a reaction array; and transferring said positionally addressable nucleic acids, polymers, oligomers or oligonucleotides from said reaction array directly to a product array to form said nucleic acid, polymer, oligomer or oligonucleotide array.

Among other things, the present invention provides for an improved method and device by which nucleic acids, nucleic acid arrays, polynucleotides, polynucleotide arrays, oligonucleotides, oligonucleotide arrays, polymers, and polymer arrays may be synthesized directly from the corresponding sub-units (e.g., by reaction of nucleotide precursors) in nanoliter volumes. Once synthesized, these polymeric or oligomeric materials may be transferred to a solid substrate to form a product array of the desired polymers or oligomers.

As a result of the small reaction volumes, reagent usage is kept to a minimum as are the attendant environmental concerns. This is particularly advantageous in large scale oligonucleotide synthesis. Furthermore, as the present invention synthesizes the desired oligomers or polymers in a positionally addressable array, the tracking, storing and indexing of a multitude of oligonucleotides is eliminated.

In a preferred embodiment, the present invention is directed to a method for producing a nucleic acid array, polymer array, or oligonucleotide array comprising: (1) synthesizing a set of polymers or oligomers on solid supports in a reaction array; and (2) transferring the polymers or oligomers from said reaction array to a product array.

A further aspect of the present invention is a device for producing a nucleic acid array, polynucleotide array, polymer array, or oligonucleotide array comprising: (1) a reaction array for solid support nucleic acid, polynucleotide, oligonucleotide, or polymer synthesis; (2) a reagent delivery module; and (3) a transfer module.

Uses of the nucleic acid, polymer or oligonucleotide arrays are further described below. These and other aspects of the present invention will be apparent to a person of skill in the art from the following description.

### BRIEF DESCRIPTION OF THE DRAWINGS

FIG. 1 illustrates a device for producing an nucleic acid, polymer, oligomer and oligonucleotide array according to one embodiment of the present invention.

### DETAILED DESCRIPTION OF THE INVENTION

Among other things, the present invention provides methods and devices which efficiently meet all of the above objectives. More particularly, the present invention provides for an improved method and device by which nucleic acids, polymers or oligonucleotides may be synthesized directly from the corresponding sub-units, (e.g., nucleosides or other nucleotide precursors) in nanoliter volumes. Once synthesized, the desired nucleic acids, polymers, or oligonucleotides are transferred to a solid substrate to form a product array of these compounds.

The methods and devices disclosed herein are generally applicable to the production of arrays comprised of diverse combinatorial chemical arrangements of nucleic acids, polynucleotides, polymers, or oligonucleotides which are immobilized on a solid substrate of a product array at known and positionally distinguishable locations to form a nucleic acid, polymer or oligonucleotide array. One preferred embodiment is an oligonucleotide array, but it is understood that this is a preferred embodiment and that other polymers are contemplated by the use of this term below.

Methods and techniques applicable to array synthesis have been described in U.S. Pat. Nos. 5,143,854, 5,242,974, 5,252,743, 5,324,633, 5,384,261, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, and 6,090,555. All of the above patents are incorporated herein by reference in their entireties for all purposes.

In a preferred embodiment, the present invention produces an array of oligonucleotides. These oligonucleotide arrays may contain any number of desired oligonucleotides for a particular experiment or use. Preferably, for most applications, the oligonucleotide arrays include from 10² or 10³ up to 10⁶ oligonucleotides arranged in any convenient matrix on a solid substrate. More preferably, the oligonucleotide arrays include from 10³ to 10⁵ oligonucleotides. Preferably, the polymers, oligonucleotides or nucleic acids occupy an area of 1 cm² for the total number recited above.

According to this embodiment of the present invention, positionally addressable oligonucleotides are synthesized in a reaction array, and are then directly transferred and immobilized on a solid substrate of a product array to form an oligonucleotide array. Generally, as described in further detail below, a device for producing an oligonucleotide array 100 according to one embodiment of the present invention comprises a reaction array 110, a product array 120, a reagent delivery module 130, and an oligonucleotide transfer module 140.

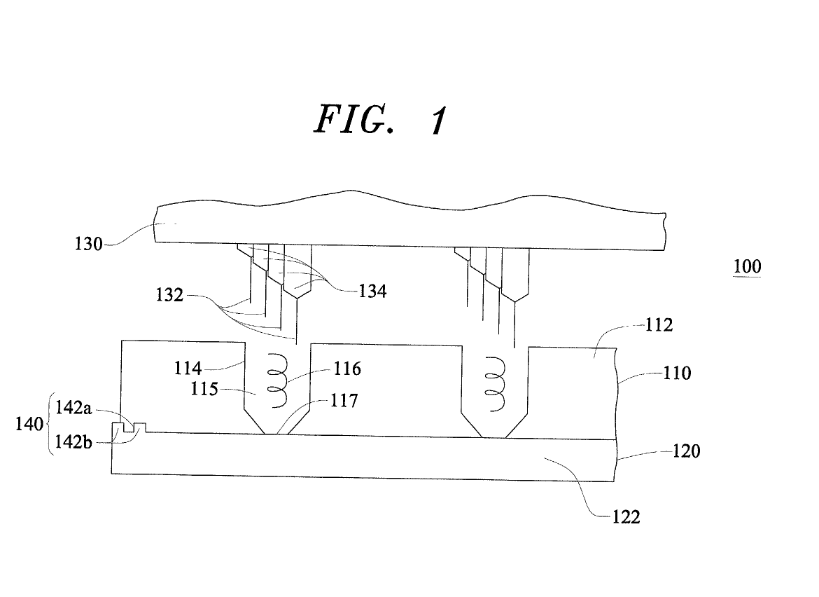

With reference to FIG. 1, the reaction array comprises a plate 112 with a plurality of reaction wells 114 therein. The reaction wells 144 may optionally include a solid reaction support 116. The reaction array thereby provides for individual reaction volumes wherein the nucleic acids, polymers, oligomers and/or oligonucleotides may be synthesized, preferably in a simultaneous fashion, while also providing positionally addressable identification of each nucleic acid, polymer, oligomer and oligonucleotide within the reaction array so that the composition of each nucleic acid, polymer, oligomer and oligonucleotide may be determined by its position within the reaction array matrix. The synthesized nucleic acids, polymers, oligomers and oligonucleotides may then be directly transferred to a product array in a manner retaining the positionally addressable identification of the nucleic acids, polymers, oligomers or oligonucleotides to thereby simplify array production and minimize the amount of reagents needed in such production.

In synthesizing nucleic acids in individual reaction volumes, it is further possible to use amplification reactions such as the polymerase chain reaction method or (PCR), the ligase chain reaction (LCR), self sustained sequence replication (3SR), strand displacement amplification (SDA) and nucleic acid based sequence amplification (NASBA), in order to amplify individual nucleic acids in their respective wells or trenches. Thereafter, small quantities of the amplification products may be transferred to a multitude of product arrays in a manner retaining the positionally addressable identification of the nucleic acids, e.g., for use in multiplex assays, to further simplify array production and minimize the amount of reagents needed in such production. Amplification may be carried out using PCR techniques that are well known in the art. See PCR Protocols: A Guide to Methods and Applications (Innis, M., Gelfand, D., Sninsky, J. and White, T., eds.) Academic Press (1990), incorporated herein by reference in its entirety for all purposes. Other amplification reactions are reviewed and disclosed in U.S. Pat. Nos. 6,238,868 and 6,218,151, which are herein incorporated by reference in their entirety for that purpose.

More particularly, the reaction wells 114 generally comprise a cavity 115 extending transversely through the plate 112 of the reaction array 110, and can optionally include a filter bottom 117 as is known in the art. The cavity 115 provides for an individual reaction volume for nucleic acid, polymer, oligomer and oligonucleotide synthesis. In one embodiment, the number of reaction wells in the reaction array may be at least as large as the number of nucleic acid, polymers, oligomers or oligonucleotides required in the final array. The reaction wells may be formed in the plate of the reaction array by any known physical technique, for example, by mechanical drilling or by laser drilling. Preferably such reaction wells are produced by a laser. The wells may be the standard well plates known in the biological industry, such as the 96 well plate. See U.S. Pat. No. 5,874,219 for well based reactions and fluid handling devices. It is incorporated by reference in its entirety.

The plate 112 of the reaction array may be comprised of any suitable material, such as glass, plastics, fluoropolymer resins (e.g., TEFLON®), aluminum, or the like. The patents referenced above and incorporated herein in their entirety teach appropriate substrates for solid supports. Preferably, the reaction array 110 comprises a plate 112 made of a fluoropolymer resin such as TEFLON® of any suitable thickness. The plate may be any suitable thickness. Preferably, the thickness of the said plate may range from about 0.1 to about 10.0 mm, and is preferably from about 0.5 to about 3 mm in thickness.

The placement of the reaction wells 114 within the reaction array 110 determines the dimensional characteristics and density of the nucleic acids, polymers, oligomers and oligonucleotides on the product array. The wells may be spaced any suitable distance. Preferably, the reaction wells are spaced between about 100 and about 1000 microns, more preferably between about 200 and about 400 microns apart form each other, as measured from the center axis of each channel. Further, the reaction wells may have a diameter of between about 50 and about 500 microns, more preferably between about 100 and about 300 microns, as measured from their widest opening. As such, the reaction well density, i.e., the number of reaction wells per square centimeter of reaction array surface, be greater than 900 holes/cm², more preferably greater than 1000 holes/cm², and still more preferably greater than 1100 holes/cm².

Again, with reference to FIG. 1, the product array 120 generally comprises a solid substrate 122 on which the positionally addressable nucleic acids, polymers, oligomers and oligonucleotides are immobilized. Methods for immobilization are known in the art and include covalent and noncovalent binding, optionally through a linker. Other methods include caged biotin binding, attachment to polylysine, or attachment to the substrate itself. The solid substrate 122 of the product array 120 may be formed from a material having a rigid or semi-rigid surface usually made from glass or suitable polymer materials known in the art. See U.S. Pat. No. 5,744,305, for example. In many embodiments, at least one surface of the solid substrate 122 is substantially flat and/or smooth, although it may be desirable to separate areas on the product array by etching the solid substrate to form raised regions or trenches and the like. See U.S. Pat. No. 6,040,193, for example. Suitable solid substrates are well known in the art and are readily commercially available through vendors such as USPG, PPG Industries, AFG Industries, as well as others described below. For instance, the solid substrate may include conventional glass; Pyrex; quartz; polymeric materials such as silicon, polystyrene, and polycarbonate; or any other suitable material known in the art.

The substrate may be biological, nonbiological, organic, inorganic, or a combination of any of these, existing as particles, strands, precipitates, gels, sheets, tubing, spheres, containers, capillaries, pads, slices, films, plates, slides, etc. The substrate may have any convenient shape, such as a disc, square, sphere, circle, etc. The substrate is preferably flat but may take on a variety of alternative surface configurations. For example, the substrate may contain raised or depressed regions on which the synthesis takes place. See U.S. Pat. No. 6,040,193.

In operation, the surface of the solid substrate may be pre-treated by cleaning with, for example, organic solvents, methylene choride, DMF, ethyl alcohol, or the like. Optionally, the substrate may be provided with appropriate linker molecules on the surface thereof. The linker molecules may be, for example, aryl acetylene, ethylene glycol oligomers containing from 2 to 20, or more specifically, 2 to 5, 7, 10, 20 monomers or more, diamines, diacids, amino acids, or combinations thereof. See U.S. Pat. No. 6,261,776. Thereafter, the surface may optionally be provided with protected surface active groups such as tertbutyloxycarbonyl (TBOC) or fluorenylmethoxycarbonyl (FMOC) protected amino acids. Such techniques are well known to those of skill in the art. See the above referenced patents for appropriate materials and techniques for manufacturing and preparing solid substrates. See U.S. Pat. Nos. 6,261,776, 5,753,788, 5,889,165, and 6,147,205 which are all hereby incorporated by reference in their entireties.

The nucleic acids, polymers, oligomers and oligonucleotides used in the arrays of the present invention may be synthesized by any known technique. In one embodiment, the nucleic acids, polymers, oligomers and oligonucleotides are synthesized on a solid reaction support 116 within the reaction well 114 prior to being transferred to the solid substrate 122 of the product array 120. The solid reaction support 116 may be comprised of a polymer, glass, other suitable material known in the art, and may be in the form of beads, fibrous matrices, or other known structures as discussed above. Generally, when the solid reaction support is in the form of beads, the bead have a diameter between about 75 and about 325 microns, preferably between about 100 and about 300 microns. In a preferred embodiment, the solid reaction support comprises control pore glass (herein after CPG) in the form of beads with a diameter of between about 75 and about 325 microns, preferably about 100 to about 300 microns. Such CPG beads generally known in the art and are available commercially.

In another embodiment, the solid reaction support 116 may be introduced into the reaction well 114 in the form of a monomer or polymer solution that can be converted to a solid polymer plug or coating by addition of a polymer initiator or by irradiation, e.g., by UV, visible, or infrared radiation. Illustrative polymers that may be used as solid reaction supports for the synthesis of nucleic acids, polymers, oligomers and oligonucleotides are generally known in the art. Methods for immobilization are known in the art and include covalent and non covalent binding, optionally through a linker. Other methods include caged biotin binding, attachment to polylysine, or attachment to the substrate itself. See the patents referenced and identified above.

The synthesis of nucleic acids, polymers, oligomers and oligonucleotides by the present invention may require functionalization of the solid reaction support surface by chemical or physical treatment, as is known in the art. See the patents referenced above. Once functionalized, chemically reactive groups on the support surface, such as carboxyl, hydroxyl, or amine groups become available to chemically bond to a linker group, which in turn can form a chemical bond to the first nucleotide or nucleotide precursor in the desired oligonucleotide sequence, for instance. The resulting solid support-linker group-nucleotide assembly will serve as the chemical platform for the solid-phase synthesis of the library of compounds which will make up the permanent array of the present invention.

In the case of glass-type solid supports discussed above, the linkage may be formed by (i) reacting the derivatized glass surface with a long chain bifunctional reagent such as, e.g., a diol, diamine, ethylene glycol oligomer, or amine-terminated ethylene glycol oligomer; (ii) reacting the free hydroxyl or amino end of the linker with the first nucleoside, which is activated as the phosphoramideite, and (iii) oxidizing of the resulting linkage to a phospotriphostriester which will be converted to a phosphate linkage after nucleic acid, polymer, oligomer or oligonucleotide synthesis is complete. In either case, the substrate-to-nucleic acid or substrate-to-polymer linkage is base stable, and the nucleic acids, polymers, oligomers or oligonucleotides will thus remain bound to the substrate throughout the deprotection steps which conclude the synthesis. See U.S. Pat. No. 6,262,216, incorporated by reference in its entirety.

Turning again to the synthesis of the desired nucleic acids, polymers, oligomers or oligonucleotide, the following is an illustrative approach which is preferably used in the synthesis of the oligonucleotides within the reaction array; however, it is of course understood that any synthetic technique known in the art may be employed in the present invention to produce the desired nucleic acids, polymers, oligomers or oligonucleotides. The patents referenced above all have significant disclosure on the reaction of monomers to form polymers. See especially U.S. Pat. Nos. 5,143,854, 5,489,678, 5,424,186, 6,261,776, 5,753,788, 5,889,165 and 6,147,205, herein incorporated by reference in their entireties. For instance, each nucleoside to be used in the synthesis is first 5′-protected, preferably by the dimethoxytrityl (hereinafter “DMT”) group. Any exocyclic amino groups on the purine and pyrimidine bases of the nucleosides are also protected, e.g., as amides according to well established methods (Gait, M. J., Ed. OLIGONUCLEOTIDE SYNTHESIS: A PRACTICAL APPROACH, Oxford University Press, Oxford, UK 1990), and can be deprotected by treatment with ammonia upon completion of the synthesis. Because the coupling reactions are sensitive to air and moisture, they are preferably carried out in an inert atmosphere.

After the first nucleoside unit is chemically secured to the linker group, a second nucleoside unit is introduced in activated form as the 3′-phosporamidite, protected at the 5′-hydroxyl by a DMT group (or another group as disclosed in the above references). The coupling reaction forms a phosphite triester linkage between the two nucleosides, which is subsequently oxidized (e.g., by iodine) to the more stable phosphotriester.

It is recommended practice in oligonucleotide synthesis (Gait, 1990, supra) to cap any unreacted 5′-hydroxyl groups remaining after each coupling step by treatment with acetic anhydride. This effectively terminates the chain and ensures that subsequent reactions proceed only by propagating chains in the desired sequences. However, this step may not be necessary if a large excess of nucleoside phosphoramidite to reactive sites is used.

After 5′-deprotection, a third similarly activated and protected nucleoside is added, resulting in a trimer after oxidation. These steps are repeated with further nucleoside units until the desired oligonucleotides have been formed within the reaction array. At this point, the terminal 5′-hydroxy groups are deprotected with dichloroacetic acid as before. Finally, the methyl groups of the phosphotriester linkages are removed by treatment with thiophenol or ammonia, and the purine and pyrimidine bases are deprotected by treatment with ammonia. (e.g., Gait, 1990, supra). These final treatment steps may, of course, be carried out simultaneously in all reaction channels of the reaction array.

The above reaction steps produce oligonucleotides having a 3′ proximal to 5′ distal orientation. Alternatively, the nucleotide subunit addition reactions may be carried out in a manner that produces 5′ proximal to 3′ distal orientation. Oligonucleotides of this type may be used for constructing position-addressable arrays of extended DNA sequence fragments, probes, or genes, for instance.

The nucleic acids, polymers, oligomers or oligonucleotides of the present invention may be synthesized with any desired number of nucleic acid residues or components. Preferably, the nucleic acid molecules made by the present process are at least 5, 10, 15, 20, 30, 40 or 50 bases long. Preferably, they are less than 250, 200, 150, 100, or 75 bases long.

If synthesis of the nucleic acids, polymers, oligomers and oligonucleotides is performed upon a solid support, the synthesis products must be cleaved from the solid support following synthesis, so that they may be transferred to the product array. Conventional chemical processes and techniques may be used to cleave the nucleic acids, polymers, oligomers and oligonucleotides from the support. For example, the linker may be cleaved photolytically, enzymatically (e.g., an ester bond), chemically (e.g., a disulfide bond) or electrically. The only limitation to the selection of the cleavable linker group is that it be resistant to the chemistry employed in synthesizing the nucleic acids, polymers, oligomers and oligonucleotides.

The reagents required for the above synthesis may be delivered by a reagent delivery module 130. In a preferred embodiment of the present invention, this system comprises four syringes 132. According to this embodiment, each syringe 132 is dedicated to the dispensing of one of the four nucleosides used in the synthesis. Additional syringes may be incorporated as needed to deliver other reagents, such as those necessary to establish or cleave the linking group, or perform any other desired chemical manipulation, such as, e.g., removal of a DMT protecting group form the 5′-hydroxyl group. Photosensitive reagents may also be used and, if so, then the appropriate light delivery system would be included.

In one embodiment, the syringes deliver a predetermined amount of the specified reagent by the advancement of a plunger 134 along a barrel of the syringe. The plunger may optionally be operated by a piezoelectric linear motor (not shown) (e.g., “Inchworm” motors, such as those available from Burleigh Instruments). Further, the movement of the syringe may be coordinated to the position of a specific reaction well or trench within the reaction array by a movable stage controlled by a computer. Such reagent delivery techniques are, for example, discussed in U.S. Pat. No. 6,040,193, the entire disclosure of which is incorporated herein by reference thereto. Further, GeneMachines, Inc. markets a 96-channel oligonucleotide synthesizer under the tradename PolyPlex™ which may be adapted for use as the reagent delivery module of the present invention.

In another embodiment of the present invention (not shown), the reagent delivery module may be a printing mechanism, which preferably comprises a thermal or piezoelectric ink-jet emitter. The ink-jet emitter in the printing mechanism operates so as to inject a small volume of the desired reagent at selected reaction channels within the reaction array, as the printing mechanism is scanned across the reaction array. A control unit, which controls the movement of the X and Y coordinates of the printer head and the emitter action of the head, can be programmed conventionally to inject initiator fluid in a selected pattern. The construction and operation of the printer head in this mode are conventional, and are known in the art.

In another embodiment of the invention (not shown), the device of the present invention further includes a multiple conduit pipetter/aspirator device for dispensing and removing substances, e.g., reagents or synthesis substrates, to and from two or more wells or trenches simultaneously or sequentially. Such apparatuses are known in the art. See, e.g., U.S. Pat. No. 6,254,826. Positive displacement plungers may also be used to draw in and expel reagents or substrates using techniques known in the art. See, e.g., U.S. Pat. No. 4,626,509.

The device of the present invention further includes a nucleotide or polymer transfer module 140. In one embodiment, the transfer module comprises a capillary contact dispenser mechanism. Such a mechanism can comprise a locking mechanism by which the solid substrate of the product array is affixed to the bottom of the reaction array (i.e., the side with the optional filter-bottom end of the reaction wells). The nucleic acids, polymers, oligomers and oligonucleotides, once cleaved from the solid support, are then allowed to contact the solid substrate by virtue of capillary action. Of course it is understood that any method of temporarily attaching the solid substrate of the product array to the bottom of the reaction array will serve as a sufficient transfer module according to the present invention. Such locking mechanisms include, for example, clamps, screws, bolts, chemical adhesives, straps, or mated locking groves etched or cut into the reaction array and the solid support of the product array, and the like.

With reference to FIG. 1, such a transfer module 140 comprises a locking mechanism 142 wherein the solid substrate 122 of the product array 120 is contacted with the reaction array 110 prior the cleavage of any linker from the optional solid reaction support 116. In the embodiment shown, the locking mechanism 142 comprises mated locking groves 142a, 142b formed in the reaction array and product array. Once the solid substrate 122 is in place, and the locking mechanism 142 is engaged, the nucleic acid, polymer, oligomer or oligonucleotide is optionally cleaved from solid support as described above and allowed to contact the solid substrate 122 of the product array 120 to thereby draw a volume of the nucleic acid, polymer, oligomer or oligonucleotide from the reaction array 110 to the product 120 through capillary action. In addition, should it be desired, the transfer of the nucleic acids, polymers, oligomers and oligonucleotides from the reaction array to the solid substrate of the product array may be facilitated by the use of pressurized gas. Such a gas may be an inert gas such as argon, nitrogen, helium, and the like, such a gas may also be compressed air.

In another embodiment (not shown), the nucleic acid, polymer, oligomer or oligonucleotide transfer module may comprise an array of pins which serve to transfer the synthesized nucleic acids, polymers, oligomers or oligonucleotides from the reaction array to the product array. Such an array of pins may generally have the same configuration as does the array of reaction wells within the reaction array. To effect the transfer of the nucleic acids, polymers, oligomers and oligonucleotides, the pins may be inserted into the individual reaction wells of the reaction array to collect samples of the nucleic acids, polymers, oligomers and oligonucleotides contained therein, and then touched to the solid support of the product array to transfer of the desired nucleic acids, polymers, oligomers and oligonucleotides to the product array. Preferably, the movement of such pins may be automated and controlled by a computer operated robot or robots, as is known in the art. See U.S. Pat. No. 6,269,846 and PCT US/01/04285 both of which are incorporated by reference in their entireties.

Of course, it is understood that depending upon the linker group chosen to connect the nucleic acids, polymers, oligomers and oligonucleotides to the permanent array, additional reagents may be needed to allow for other reactions to proceed which are necessary to effect the attachment of the linker present on the terminus of the nucleic acid, polymer, oligomer or oligonucleotide to the functional surface of the solid substrate of the permanent array. Further, as described above, the solid substrate of the product array may be functionalized prior to transfer of the nucleic acids, polymers, oligomers or oligonucleotides. In this manner the cleaved nucleic acids, polymers, oligomers or oligonucleotides may be chemically bound to the solid substrate of the product to form an array according to the present invention.

Once transfer of the nucleic acids, polymers, oligomers or oligonucleotides to the product array is complete, the solid substrate may be removed from the reaction array to obtain the nucleic acid, polymer, oligomer or oligonucleotide array. The nucleic acids, polymers, oligomers and oligonucleotides so transferred to the product array are immobilized thereon with good bio-accessibility. Such DNA arrays enable highly parallel hybridization experiments for use in the discovery of novel therapeutics and diagnostics, as well as for diagnostic screening of populations and patient groups. Such applications include gene expression, re-sequencing, and genotyping for discovery and polymorphism detection for pharmacogenetics, screening and diagnostics. See U.S. Pat. No. 5,800,992 which is also incorporated by reference in its entirety for all purposes.

The technology provided by the present invention has very broad applications. In particular, the present invention may be used to completely sequence a given target sequence to subunit resolution. This may be for de novo sequencing, or may be used in conjunction with a second sequencing procedure to provide independent verification. See, e.g., (1988) Science 242:1245. For example, a large polynucleotide sequence defined by either the Maxam and Gilbert technique or by the Sanger technique may be verified by using the present invention.

In addition, by selection of appropriate probes, a polynucleotide sequence can be fingerprinted. Fingerprinting is a less detailed sequence analysis which usually involves the characterization of a sequence by a combination of defined features. Sequence fingerprinting is particularly useful because the repertoire of possible features which can be tested is virtually infinite. Moreover, the stringency of matching is also variable depending upon the application. A Southern Blot analysis may be characterized as a means of simple fingerprint analysis.

Fingerprinting analysis may be performed to the resolution of specific nucleotides, or may be used to determine homologies, most commonly for large segments. In particular, an array of oligonucleotide probes of virtually any workable size may be positionally localized on a matrix and used to probe a sequence for either absolute complementary matching, or homology to the desired level of stringency using selected hybridization conditions.

In addition, the present invention provides means for mapping analysis of a target sequence or sequences. Mapping will usually involve the sequential ordering of a plurality of various sequences, or may involve the localization of a particular sequence within a plurality of sequences. This may be achieved by immobilizing particular large segments onto the matrix and probing with a shorter sequence to determine which of the large sequences contain that smaller sequence. Alternatively, relatively shorter probes of known or random sequence may be immobilized to the matrix and a map of various different target sequences may be determined from overlaps.

Principles of such an approach are described in some detail by Evans et al. (1989) “Physical Mapping of Complex Genomes by Cosmid Multiplex Analysis,” Proc. Natl. Acad. Sci. USA 86:5030-5034; Michiels et al. (1987) “Molecular Approaches to Genome Analysis: A Strategy for the Construction of Ordered Overlap Clone Libraries,” CABIOS 3:203-210; Olsen et al. (1986) “Random-Clone Strategy for Genomic Restriction Mapping in Yeast,” Proc. Natl. Acad. Sci. USA 83:7826-7830; Craig, et al. (1990) “Ordering of Cosmid Clones Covering the Herpes Simplex Virus Type I (HSV-I) Genome: A Test Case for Fingerprinting by Hybridization,” Nuc. Acids Res. 18:2653-2660; and Coulson, et al. (1986) “Toward a Physical Map of the Genome of the Nematode Caenorhabditis elegans,” Proc. Natl. Acad. Sci. USA 83:7821-7825; each of which is hereby incorporated herein by reference.

Fingerprinting analysis also provides a means of identification. In addition to its value in apprehension of criminals from whom a biological sample, e.g., blood, has been collected, fingerprinting can ensure personal identification for other reasons. For example, it may be useful for identification of bodies in tragedies such as fire, flood, and vehicle crashes. In other cases the identification may be useful in identification of persons suffering from amnesia, or of missing persons. Other forensics applications include establishing the identity of a person, e.g., military identification “dog tags”, or may be used in identifying the source of particular biological samples. Fingerprinting technology is described, e.g., in Carrano, et al. (1989) “A High-Resolution, Fluorescence-Based, Semi-automated method for DNA Fingerprinting,” Genomics 4: 129-136, which is hereby incorporated herein by reference.

The fingerprinting analysis may be used to perform various types of genetic screening. For example, a single substrate may be generated with a plurality of screening probes, allowing for the simultaneous genetic screening for a large number of genetic markers. Thus, prenatal or diagnostic screening can be simplified, economized, and made more generally accessible.

Other analyses applications may be performed, e.g., sample extraction, amplification reactions, nucleic acid fragmentation and labeling, extension reactions, transcription reactions and the like, by depositing a subset of the substrates into a diagnostic system designed for multiplex assays. For example, such a diagnostic device may further include a reader device for scanning and obtaining the data from the device, and a computer based interface for controlling the device and/or interpretation of the data derived from the device. A variety of amplification methods are suitable for use in the methods and device of the present invention, including for example, the polymerase chain reaction method or (PCR), the ligase chain reaction (LCR), self sustained sequence replication (3SR), strand displacement amplification (SDA) and nucleic acid based sequence amplification (NASBA). U.S. Pat. No. 6,197,595 discloses methods of integrating nucleic acid arrays with amplification diagnostic devices, and is herein incorporated by reference in its entirety.

All publications, patent applications, and patents cited in this specification are incorporated by reference in their entirety where they are cited, and are also cited as indicative of the skill in the art and available general knowledge.

While this invention can be described in connection with what is presently considered to be practical and preferred embodiments, it should be understood that it is not to be limited or restricted to the disclosed embodiments but, on the contrary, is intended to cover various modifications, substitutions, and combinations within the scope of the appended claims. In this respect, one should note that the protection conferred by the claims is determined after their issuance in view of later technical developments and would extend to all legal equivalents.

Therefore, it is to be understood that variations in this invention that are not described herein would be obvious to a person skilled in the art and could be practiced without departing from the invention's novel and non-obvious elements with the proviso that the prior art is excluded.

What is claimed:

1. A method for producing nucleic acid, polymer, oligomer or oligonucleotide array comprising:
a) synthesizing a set of positionally addressable nucleic acids, polymers, oligomers or oligonucleotides in a reaction array; and
b) transferring said positionally addressable nucleic acids, polymers, oligomers or oligonucleotides from said reaction array directly to a product array to form said nucleic acid, polymer, oligomer or oligonucleotide array.

2. A method for producing an nucleic acid, polymer, oligomer or oligonucleotide array comprising:
a) synthesizing a set of positionally addressable nucleic acids, polymers, oligomers or oligonucleotides on solid reaction supports in a reaction array;
b) cleaving said positionally addressable nucleic acids, polymers, oligomers or oligonucleotides from said solid reaction supports; and
c) transferring said cleaved positionally addressable nucleic acids, polymers, oligomers or oligonucleotides from said reaction array directly to a product array to form said nucleic acid, polymer, oligomer or oligonucleotide array;
wherein said reaction array comprises a plate with a plurality reaction wells.

3. The method of claim 2 wherein said solid reaction support is comprised of a material selected from the group consisting of controlled pore glass, a polymer, and a polymer suspension.

4. The method of claim 2 wherein said plate of said reaction array is comprised of plastic.

5. The method of claim 2 wherein said plate of said reaction array is comprised of a fluoropolymer resin.

6. The method of claim 2 wherein said plate of said reaction array is between about 0.1 and about 10.0 mm in thickness.

7. The method of claim 2 wherein said reaction wells have a maximum diameter of between about 50 and about 500 microns.

8. The method of claim 2 wherein said reaction wells are spaced between about 100 and about 1000 microns apart from their respective centers.

9. The method of claim 2 wherein said plate of said reaction array has a reaction well density greater than 900 holes per cm².

10. The method of claim 2 wherein said plate of said reaction array has a reaction well density greater than 1000 holes per cm².

11. The method of claim 2 wherein said plate of said reaction array has a reaction well density greater than 1100 holes per cm².

12. The method of claim 2 wherein said nucleic acids, polymers, oligomers or oligonucleotides are synthesized by sequential introduction of reagents from an automated dispensing system.

13. The method of claim 12 wherein said automatic dispensing system is comprised of four syringes.

14. The method of claim 12 wherein said reagents are amidites.

15. The method of claim 12 wherein said automated dispensing system is comprised of an ink-jet emitter.

16. The method of claim 2 wherein said nucleic acids, polymers, oligomers or oligonucleotides are transferred from said reaction array directly to said product array via robotically controlled transfer pins.

17. The method of claim 2 wherein said nucleic acids, polymers, oligomers or oligonucleotides are transferred from said reaction array directly to said product array via direct contact between the product array and the reaction array.

18. The method of claim 17 wherein the said transfer is facilitated by the use of a pressurized inert gas.

19. A device for producing an nucleic acid, polymer, oligomer or oligonucleotide array comprising:
a) a reaction array for nucleic acid, polymer, oligomer or oligonucleotide synthesis comprising a plate with a plurality of reaction wells;
b) a reagent delivery module;
c) an nucleic acid, polymer, oligomer or oligonucleotide transfer module; and
d) a product array comprising a solid substrate.

20. The device of claim 19 wherein said plurality of reaction wells further comprises solid reaction supports.

21. The device of claim 20 wherein said solid reaction support is comprised of a material selected from the group consisting of controlled pore glass a polymer, and a polymer suspension.

22. The device of claim 19 wherein said plate of said reaction array is comprised of plastic.

23. The device of claim 19 wherein said plate of said reaction array is comprised of a fluoropolymer resin.

24. The device of claim 19 wherein said plate of said reaction array is between about 0.1 and about 10.0 mm in thickness.

25. The device of claim 19 wherein said reaction wells have a maximum diameter of between about 500 and about 500 microns.

26. The device of claim 19 wherein said reaction wells are spaced between about 100 and about 1000 microns apart from their respective centers.

27. The device of claim 19 wherein said plate of said reaction array has a reaction well density greater than 900 holes per cm².

28. The device of claim 19 wherein said plate of said reaction array has a reaction well density greater than 1000 holes per cm².

29. The device of claim 19 wherein said plate of said reaction array has a reaction well density greater than 1100 holes per cm².

30. The device of claim 19 further comprising an automated synthesis reagent dispensing system.

31. The device of claim 30 wherein said automated synthesis reagent dispensing system comprises four syringes.

32. The device of claim 30 wherein said automated synthesis reagent dispensing system comprises an ink-jet emitter.

33. The device of claim 19 wherein said nucleic acid, polymer, oligomer or oligonucleotide transfer module comprises an array of transfer pins.

34. The device of claim 33 further comprising a robotic control module for controlling said transfer pins.

35. The device of claim 19 wherein said nucleic acid, polymer, oligomer or oligonucleotide transfer module comprises a clamp attaching said product array to the bottom of said reaction array.

36. The device of claim 19 wherein said nucleic acid, polymer, oligomer or oligonucleotide transfer module comprises a bolt attaching said product array to the bottom of said reaction array.

37. The device of claim 19 wherein said nucleic acid, polymer, oligomer or oligonucleotide transfer module comprises a pressurized inert gas dispenser for facilitating transfer of the nucleic acids, polymers, oligomers and oligonucleotides from the reaction array to the product array.

38. A method for producing a polymer array comprising:
a) synthesizing a set of positionally addressable polymers in a reaction array; and
b) transferring said positionally addressable polymers from said reaction array directly to a product array to form said polymer array.

39. A method for producing an polynucleotide array comprising:
a) synthesizing a set of positionally addressable polynucleotides in a reaction array; and
b) transferring said positionally addressable polynucleotides from said reaction array directly to a product array to form said polynucleotide array.
